(19) United States
(12) Patent Application Publication
(10) Pub. No.: US 2018/0305433 A1
(43) Pub. Date: Oct. 25, 2018

(54) Cell

(71) Applicant: AUTOLUS LIMITED, London (GB)

(72) Inventors: Martin Pulé, London (GB); Shaun Cordoba, London (GB)

(21) Appl. No.: 15/770,130

(22) PCT Filed: Oct. 21, 2016
(86) PCT No.: PCT/GB2016/053291

§ 371 Date: Apr. 20, 2018

(30) Foreign Application Priority Data
Oct. 23, 2015 (GB) 1518817.0

(51) Int. Cl. C07K 14/725; A61K 35/17; C07K 16/28; C07K 14/705; C12N 5/0783; C12N 9/16

(52) U.S. Cl. CPC C07K 14/7051; A61K 35/17; C07K 16/2896; C07K 14/70517; A61K 39/00; C12N 5/0638; C12N 9/16; C12Y 301/03048; C12N 2510/00; C07K 16/2803

(57) ABSTRACT

The present invention relates to a cell which co-expresses a first chimeric antigen receptor (CAR) and second CAR at the cell surface, each CAR comprising: (i) an antigen-binding domain; (ii) a spacer (iii) a trans-membrane domain; and (iv) an endodomain wherein the first CAR is an activating CAR comprising an activating endodomain and the second CAR is an inhibitory CAR comprising a ligation-off inhibitory endodomain; wherein the first CAR binds to a first antigen and the second CAR binds to a second antigen, wherein both the first and second antigens are expressed on the surface of a target cell; wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and wherein the spacer of the first CAR is an equivalent size to the spacer of the second CAR.

### FIELD OF THE INVENTION

The present invention relates to a cell which comprises more than one chimeric antigen receptor (CAR). The cell may be capable of specifically recognising a target cell, due to a differential pattern of expression of two or more antigens by the target cell.

### BACKGROUND TO THE INVENTION

A number of immunotherapeutic agents have been described for use in cancer treatment, including therapeutic monoclonal antibodies (mAbs), immunoconjugated mAbs, radioconjugated mAbs and bi-specific T-cell engagers.

Typically these immunotherapeutic agents target a single antigen: for instance, Rituximab targets CD20; Myelotarg targets CD33; and Alemtuzumab targets CD52.

However, it is relatively rare for the presence (or absence) of a single antigen effectively to describe a cancer, which can lead to a lack of specificity.

Most cancers cannot be differentiated from normal tissues on the basis of a single antigen. Hence, considerable “on-target off-tumour” toxicity occurs whereby normal tissues are damaged by the therapy. For instance, whilst targeting CD20 to treat B-cell lymphomas with Rituximab, the entire normal B-cell compartment is depleted, whilst targeting CD52 to treat chronic lymphocytic leukaemia, the entire lymphoid compartment is depleted, whilst targeting CD33 to treat acute myeloid leukaemia, the entire myeloid compartment is damaged etc.

The predicted problem of “on-target off-tumour” toxicity has been borne out by clinical trials. For example, an approach targeting ERBB2 caused death to a patient with colon cancer metastatic to the lungs and liver. ERBB2 is over-expressed in colon caner in some patients, but it is also expressed on several normal tissues, including heart and normal vasculature.

For some cancers, targeting the presence of two cancer antigens may be more selective and therefore effective than targeting one. For example, B-chronic lymphocytic leukaemia (B-CLL) is a common leukaemia which is currently treated by targeting CD19. This treats the lymphoma but also depletes the entire B-cell compartment such that the treatment has a considerable toxic effect. B-CLL has an unusual phenotype in that CD5 and CD19 are co-expressed. By targeting only cells which express CD5 and CD19, it would be possible to considerably reduce on-target off-tumour toxicity.

There is thus a need for immunotherapeutic agents which are capable of more targeting to reflect the complex pattern of marker expression that is associated with many cancers.

### Chimeric Antigen Receptors (CARs)

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 2a).

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

However, the use of CAR-expressing T cells is also associated with on-target, off tumour toxicity. For example, a CAR-based approach targeting carboxy anyhydrase-IX (CAIX) to treat renal cell carcinoma resulted in liver toxicity which is thought to be caused by the specific attack on bile duct epithelial cells (Lamers et al (2013) Mol. Ther. 21:904-912).

### Dual Targeting CAR Approaches

In order to address the problem of “on target, off tumour” toxicity, CAR T cells have been developed with dual antigen specificity. In the “dual targeting” approach, two complementary CARs are co-expressed in the same T-cell population, each directed to a distant tumour target and engineered to provide complementary signals.

WIikie et al (2012 J Clin Immunol 32:1059-1070) describe a dual targeting approach in which ErbB2- and MUC1-specific CARs are co-expressed. The ErbB2-specific CAR provided the CD3ζ signal only and the MUC1-specific CAR provided the CD28 co-stimulatory signal only. It was found that complementary signalling occurred in the presence of both antigens, leading to IL-2 production. However, IL-2 production was modest when compared to control CAR-engineered T cells in which signaling is delivered by a fused CD28+CD3ζ endodomain.

A similar approach was described by Kloss et al (2013 Nature Biotechnol. 31:71-75) in which a PSCA specific CAR was used which provides a CD3-mediated activation signal in combination with a chimeric co-stimulatory receptor specific for PSMA. With this ‘co-CAR’ design, the CAR T-cell receives an activation signal when it encounters a target cell with one antigen, and a co-stimulatory signal when it encounters a target cell with the other antigen, and only receives both activatory and co-stimulatory signals upon encountering target cells bearing both antigens.

This represents an early attempt at restricting CAR activity to only a target cell bearing two antigens. This approach however is limited: although CAR T-cell activity will be greatest against targets expressing both antigens, CAR T-cells will still kill targets expressing only antigen recognized by the activatory CAR; further, co-stimulation results in prolonged effects on T-cells which last long after release of target cell. Hence, activity against single-antigen positive T-cells equal to that against double-positives might be possible for example in a situation where single-positive tissues are adjacent to, or in a migratory path from double positive tumour.

### CAR Logical AND Gates

In WO2015/075469, the present inventors describe a revolutionary new system for targeting two or more antigens, which utilizes the physiological mechanism of kinetic segregation which takes place at the T-cell/target cell interface.

Kinetic-segregation is the mechanism by which antigen recognition by an antigen receptor is converted into down-stream activation signals (FIG. 1). Briefly: at the ground (“off”) state, the signalling components on the T-cell membrane are in dynamic equilibrium whereby dephosphorylated ITAMs are favoured. This is due to the greater expression level of the highly potent transmembrane CD45/CD148 phosphatase over membrane-tethered kinases such as Lck. When a T-cell engages a target cell through a T-cell receptor (or CAR) a tight immunological synapse forms. This close juxtapositioning of the T-cell and target membranes passively excludes CD45/CD148 due to their large ectodomains which cannot fit into the synapse. Aggregation of a high concentration of ITAM associated receptors and the lack of segregation of kinases in the synapse, in the absence of phosphatases, leads to a state whereby phosphorylated ITAMs are favoured. This is the “on” state. ZAP70 now recognizes a threshold of phosphorylated ITAMs and propagates a T-cell activation signal.

The present inventors have previously shown that, where a cell expresses two CARs recognising separate antigens, it is possible to cause the two CARs to dyssegregate by using spacers of a different size.

In the CAR logical “AND” gate, one of the first or second CARs is an activating CAR comprising an activating endodomain, and the other CAR is a “ligation-off” inhibitory CAR comprising an inhibitory endodomain. The ligation-off inhibitory CAR inhibits T-cell activation by the activating CAR in the absence of inhibitory CAR ligation, but does not significantly inhibit T-cell activation by the activating CAR when the inhibitory CAR is ligated.

In the system described in WO2015/075469 the spacer of the first and second CAR are of a different size, such that when both CARs are ligated, they segregate. This causes the inhibitory CAR to be spatially separated from the activating CAR, so that T cell activation can occur. T cell activation therefore only occurs in response to a target cell bearing both cognate antigens.

### DESCRIPTION OF THE FIGURES

FIG. 1—Cartoon of kinetic segregation model of CAR T-cell activation.

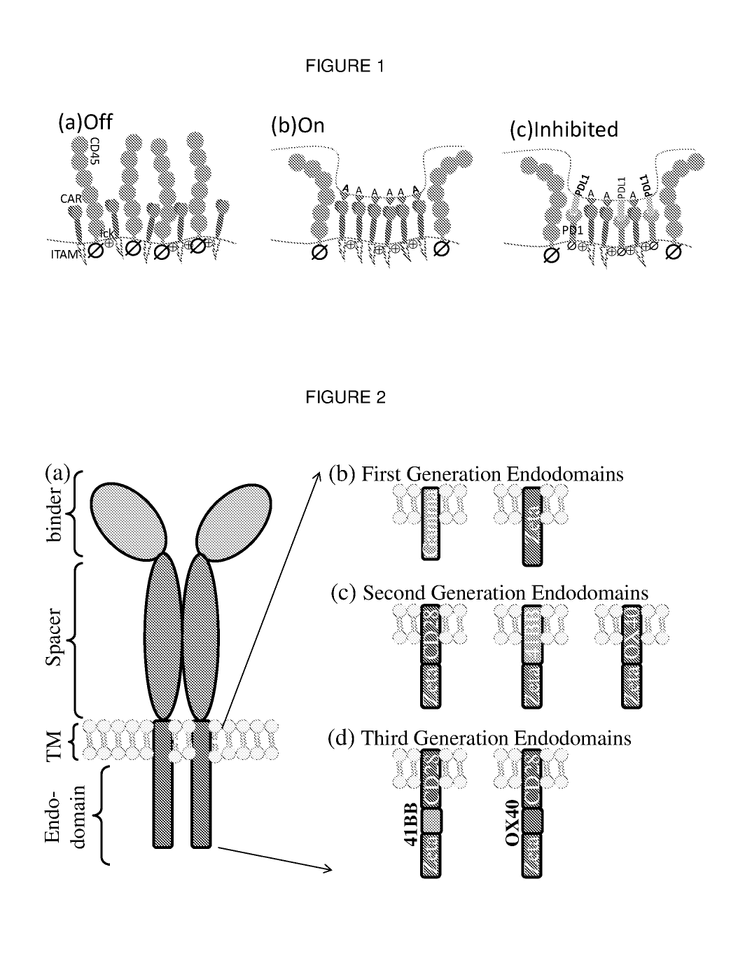

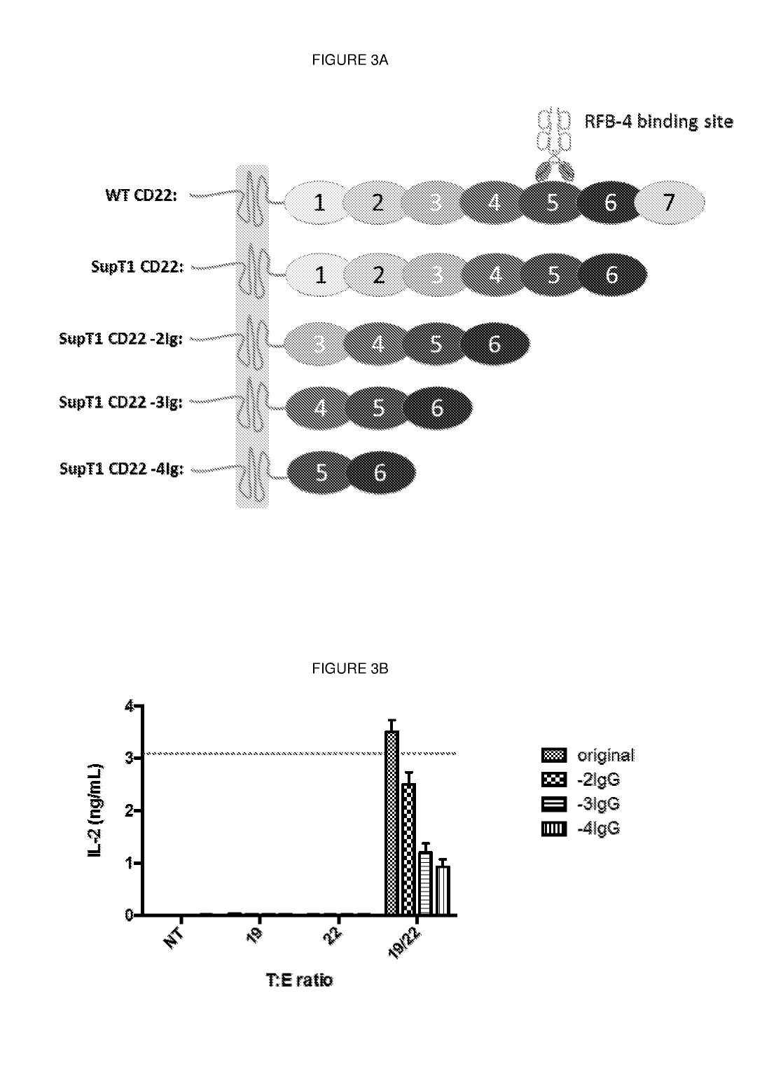

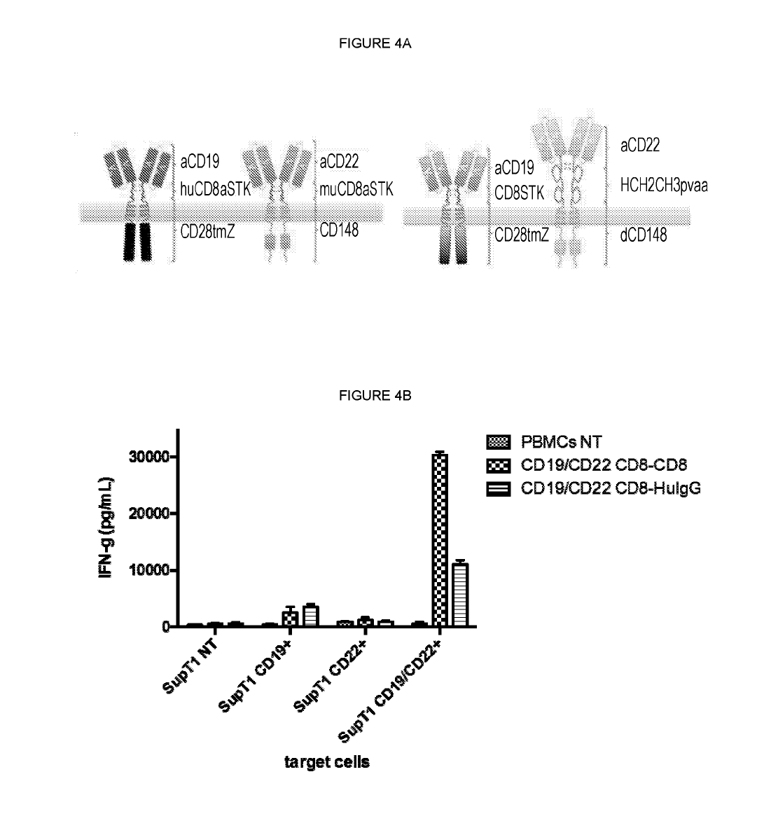

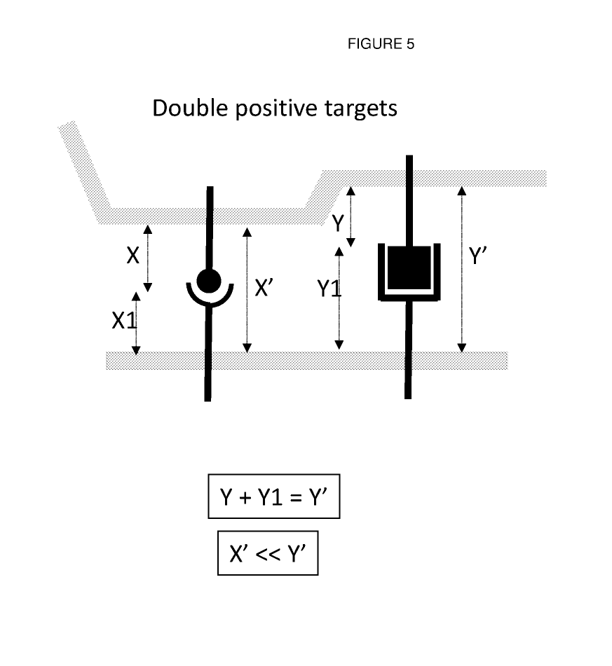

The T-cell membrane is shown in red while the target cell is shown in blue. The space between both membranes represents the extracellular space between T-cell and target cell where an immunological synapse may form. CARs (shown in blue) contain ITAMs in their endodomains. CD45 (shown in grey) have bulky ectodomains and a potent phosphatase as endodomain (denoted by a large “Ø”). Lck is tethered to the membrane and is denoted by a “⊕”. (a) In the “off” state, presence of powerful phosphatases from CD45 (and CD148) favour dephosphorylation of ITAMs. (b) The “on” state occurs when CAR recognizes its cognate antigen on a target cell. An immunological synapse forms and the bulky ectodomains of CD45/CD148 result in their exclusion from the synapse. Now phosphorylated ITAMs are favoured. (c) The “inhibited” state occurs when inhibitory immune receptors (here PD1 as an example is denoted in green) recruit phosphatases like SHP1 (PTPN6) into a synapse resulting in dephosphorylation of ITAMs despite cognate antigen recognition.

FIG. 2—(a) Generalized architecture of a CAR: A binding domain recognizes antigen; the spacer elevates the binding domain from the cell surface; the trans-membrane domain anchors the protein to the membrane and the endodomain transmits signals. (b) to (d): Different generations and permutations of CAR endodomains: (b) initial designs transmitted ITAM signals alone through FcεR1-γ or CD3 endodomain, while later designs transmitted additional (c) one or (d) two co-stimulatory signals in cis.

FIG. 3—Investigating the effect of relative distance of the epitopes from the target cell membrane on AND gate function

A. A set of target cells were created expressing a truncation series of CD22. A series of deletion mutants were created lacking Ig domains 1 and 2 (CD22-2Ig); Ig domains 1, 2 and 3 (CD22-3Ig); or Ig domains 1, 2, 3 and 4 (CD22-4Ig), bringing the RFB-4 binding epitope incrementally closer to the target cell membrane.

B. Testing AND gate function. CAR T cells were created expressing an anti-CD19 activating CAR with a CD8 stalk spacer and a CD3 Zeta endodomain; and an anti-CD22 inhibitory CAR with an RFB-4-based antigen-binding domain, an Fc spacer and a CD148 endodomain. AND gate function was tested in T cells and it was shown that the CAR pair functioned as an AND gate for all truncated CD22 variants but AND gate function was improved by increasing the distance of the RFB-4 binding epitope from the target cell membrane.

FIG. 4—Testing AND gate function with different spacer pairs.

A. CAR T cells were created expressing 1) and anti-CD19 activating CAR with a human CD8 stalk spacer and a CD3 Zeta endodomain, and 2) an anti-CD22 inhibitory CAR with an RFB-4-based antigen-binding domain, a murine CD8 stalk spacer and a CD148 endodomain (FIG. 4A, left-hand figure).

CAR T cells were also created expressing 1) and anti-CD19 activating CAR with a CD8 stalk spacer and a CD3 Zeta endodomain, and 2) an anti-CD22 inhibitory CAR with an RFB-4-based antigen-binding domain, a Ig Fc spacer and a CD148 endodomain (FIG. 4A, right-hand figure).

B. The AND gate function of these CAR T cells was compared using target cells expressing CD19 alone, CD22 alone, or both antigens. Both CAR pairs functioned as an AND gate. However, for this CD19/CD22 target antigen pair where there is a significant difference in spacing of the epitopes from the target cell membrane, AND gate function was improved using a pair of CARs with similar sized spacers.

FIG. 5—Schematic diagram illustrating the various distance parameters at the T-cell:target cell synapse.

### SUMMARY OF ASPECTS OF THE INVENTION

The present inventors have now found a new approach for making a CAR logical AND gate.

They have found that by designing the CARs such that one CAR recognises an epitope which is relatively proximal to the membrane of the target cell, whereas the other CAR recognises an epitope which is relatively distal to the membrane of the target cell, it is possible to create a functional AND gate, even with similarly sized spacers.

Surprisingly, where there is a choice of:

A. causing dys-segregation by difference in spacer length (using the system described in WO2015/075469); or
B. causing dys-segregation by difference in the relative distance of the epitope of the two antigens from the cell membrane, using the system of the present invention, AND gate function is better using system B.

Thus, in a first aspect, the present invention provides a cell which co-expresses a first chimeric antigen receptor (CAR) and second CAR at the cell surface, each CAR comprising:
(i) an antigen-binding domain;
(ii) a spacer
(iii) a trans-membrane domain; and
(iv) an endodomain
   wherein the first CAR is an activating CAR comprising an activating endodomain and the second CAR is an inhibitory CAR comprising a ligation-off inhibitory endodomain;
   wherein the first CAR binds to a first antigen and the second CAR binds to a second antigen, wherein both the first and second antigens are expressed on the surface of a target cell;
   wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
   wherein the spacer of the first CAR is an equivalent size to the spacer of the second CAR.

When the first CAR and the second CAR bind their respective target antigens,

(X+X1)«(Y+Y1)

where:
X is the distance from the target cell membrane to the epitope on the first antigen;
X1 is the length of the first CAR;
Y is the distance from the target cell membrane to the epitope on the second antigen;
Y1 is the length of the second CAR;
such that the first CAR and second CAR become spatially separated on the immune cell membrane.

In the formula given above, X may be less than 8 nm and Y may be greater than 15 nm.

The spacer of the first CAR and the spacer of the second CAR may be orthologous.

The inhibitory endodomain may comprise all or part of the endodomain from CD148 or CD45.

The cell may be a T-cell or a natural killer (NK) cell.

In a second aspect, the present invention provides a nucleic acid sequence encoding both the first and second chimeric antigen receptors (CARs) as defined in the first aspect of the invention.

The nucleic acid sequence may have the following structure:

AgB1-spacer1-TM1-endo1-coexpr-AbB2-spacer2-TM2-endo2
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the endodomain of the first CAR;
coexpr is a nucleic acid sequence enabling co-expression of both CARs AgB2 is a nucleic acid sequence encoding the antigen-binding domain of the second CAR;
spacer 2 is a nucleic acid sequence encoding the spacer of the second CAR; TM2 is a nucleic acid sequence encoding the transmembrane domain of the second CAR;
endo 2 is a nucleic acid sequence encoding the endodomain of the second CAR; which nucleic acid sequence, when expressed in a cell, encodes a polypeptide which is cleaved at the cleavage site such that the first and second CARs are co-expressed at the cell surface.

The “coexpr” may encode a sequence comprising a self-cleaving peptide.

Alternative codons may be used in regions of sequence encoding the same or similar amino acid sequences, in order to avoid homologous recombination.

In a third aspect, the present invention provides a kit which comprises
(i) a first nucleic acid sequence encoding the first chimeric antigen receptor (CAR) as defined in the first aspect of the invention, which nucleic acid sequence has the following structure:
   AgB1-spacer1-TM1-endo1
   in which
   AgB1 is a nucleic acid sequence encoding the antigen-binding domain of the first CAR;
   spacer 1 is a nucleic acid sequence encoding the spacer of the first CAR;
   TM1 is a nucleic acid sequence encoding the transmembrane domain of the first CAR;
   endo 1 is a nucleic acid sequence encoding the endodomain of the first CAR; and
(ii) a second nucleic acid sequence encoding the second chimeric antigen receptor (CAR) as defined in any of claims 1 to 5, which nucleic acid sequence has the following structure:
   AgB2-spacer2-TM2-endo2
   AgB2 is a nucleic acid sequence encoding the antigen-binding domain of the second CAR;
   spacer 2 is a nucleic acid sequence encoding the spacer of the second CAR;
   TM2 is a nucleic acid sequence encoding the transmembrane domain of the second CAR;
   endo 2 is a nucleic acid sequence encoding the endodomain of the second CAR.

There is also provided a kit comprising: a first vector which comprises the first nucleic acid sequence as defined in the second aspect of the invention; and a second vector which comprises the first nucleic acid sequence as defined in the second aspect of the invention.

The vectors may, for example, be integrating viral vectors or transposons.

In a fourth aspect there is provided a vector comprising a nucleic acid sequence according to the second aspect of the invention.

The vector may, for example, be a retroviral vector or a lentiviral vector or a transposon.

In a fifth aspect there is provided a method for making a cell according to the first aspect of the invention, which comprises the step of introducing: a nucleic acid sequence according to the second aspect of the invention; a first nucleic acid sequence and a second nucleic acid sequence as defined in the third aspect of the invention; and/or a first vector and a second vector as defined in the third aspect of the invention or a vector according to the fourth aspect of the invention, into a cell.

The cell may be from a sample isolated from a subject.

In a sixth aspect there is provided a pharmaceutical composition comprising a plurality of cells according to the first aspect of the invention.

In a seventh aspect there is provided a method for treating and/or preventing a disease, which comprises the step of administering a pharmaceutical composition according to the sixth aspect of the invention to a subject.

The method may comprise the following steps:
(i) isolation of a cell-containing sample from a subject;
(ii) transduction or transfection of the cells with: a nucleic acid sequence according to the second aspect of the invention; a first nucleic acid sequence and a second nucleic acid sequence as defined in the third aspect of the invention; and/or a first vector and a second vector as defined in the third aspect of the invention or a vector according to the fourth aspect of the invention; and
(iii) administering the cells from (ii) to a the subject.

The disease may be a cancer.

There is also provided pharmaceutical composition according to the sixth aspect of the invention for use in treating and/or preventing a disease.

There is also provided the use of a cell according to the first aspect of the invention in the manufacture of a medicament for treating and/or preventing a disease.

### DETAILED DESCRIPTION

CHIMERIC ANTIGEN RECEPTORS (CARs)

CARs, which are shown schematically in FIG. 2, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen.

The present invention relates to a cell which co-expresses a first CAR and a second CAR such that a T-cell can recognize expression of both antigens on target cells in the manner of a logic gate as detailed in truth table 1 below.

Both the first and second (and optionally subsequent) CARs comprise:

(i) an antigen-binding domain;
(ii) a spacer;
(iii) a transmembrane domain; and
(iii) an intracellular domain.

TABLE 1
Truth Table for CAR AND GATE

| Antigen A | Antigen B | Response |
|---|---|---|
| Absent | Absent | No activation |
| Absent | Present | No Activation |
| Present | Absent | No Activation |
| Present | Present | Activation |

The first and second CAR of the cell of the present invention may be produced as a polypeptide comprising both CARs, together with a cleavage site.

SEQ ID No. 1 gives an example of such a polypeptide, which comprises two CARs.

SEQ ID No 1 is a CAR AND gate which recognizes CD19 AND CD22 using a CD148 phosphatase

MSLPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITKAGGGGSGGGGSGGGGSGGGGS
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSDPTTTPAPRPPTPAPTIASQPLSLRPEACR
PAAGGAVHTRGLDFACDIFWVLVVVGGVLACYSLLVTVAFIIFWVRRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRRAEGRGSLLTCGDVEENPGPMEFGLSWLFLVAILKGVQCEVQ
LVESGGGLVQPGGSLRLSCAASGFAFSIYDMSWVRQVPGKGLEWVSYISS
GGGTTYYPDTVKGRFTISRDNSRNTLDLQMNSLRVEDTAVYYCARHSGYG
SSYGVLFAYWGQGTLVTVSSGGGGSGGGGSGGGGSDIQMTQSPSSLSASV
GDRVTITCRASQDISNYLNWLQQKPGKAPKLLIYYTSILHSGVPSRFSGS
GSGTEFTLTISSLQPEDFATYYCQQGNTLPWTFGQGTKLEIKRSDPAEPK
SPDKTHTCPPCPAPPVAGPSVFLFPPKPKDTLMIARTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQ
GNVFSCSVMHEALHNHYTQKSLSLSPGKKDPKAVFGCIFGALVIVTVGGF
IFWRKKRKDAKNNEVSFSQIKPKKSKLIRVENFEAYFKKQQADSNCGFAE
EYEDLKLVGISQPKYAAELAENRGKNRYNNVLPYDISRVKLSVQTHSTDD
YINANYMPGYHSKKDFIATQGPLPNTLKDFWRMVWEKNVYAIIMLTKCVE
QGRTKCEEYWPSKQAQDYGDITVAMTSEIVLPEWTIRDFTVKNIQTSESH
PLRQFHFTSWPDHGVPDTTDLLINFRYLVRDYMKQSPPESPILVHCSAGV
GRTGTFIAIDRLIYQIENENTVDVYGIVYDLRMHRPLMVQTEDQYVFLNQ
CVLDIVRSQKDSKVDLIYQNTTAMTIYENLAPVTTFGKTNGYIA

The various components of this polypeptide (aCD19fmc63-CD8STK-CD28tmZ-2A-aCD22hum-HCH2CH3pvaa-dCD148) break down as follows:

signal peptide aCD19 CAR
SEQ ID No. 16
MSLPVTALLLPLALLLHA
aCD19 heavy
SEQ ID No. 17
ARPDIQMTQTTSSLSASLGDRVTISCRASQDISKYLNWYQQKPDGTVKLL
IYHTSRLHSGVPSRFSGSGSGTDYSLTISNLEQEDIATYFCQQGNTLPYT
FGGGTKLEITKAGGGG
linker
SEQ ID No. 18
SGGGGSGGGGSGGGGS
aCD19 light
SEQ ID No. 19
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSD
CD8STK
SEQ ID No. 20
PTTTPAPRPPTPAPTIASQPLSLRPEACRPAAGGAVHTRGLDFACDI
CD28tm
SEQ ID No. 21
FWVLVVVGGVLACYSLLVTVAFIIFWV
TCRz
SEQ ID No. 22
RRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
2A
SEQ ID No. 15
RAEGRGSLLTCGDVEENPGP
signal peptide aCD22 CAR
SEQ ID No. 23
MEFGLSWLFLVAILKGVQCE
aCD22-heavy
SEQ ID No. 24
VQLVESGGGLVQPGGSLRLSCAASGFAFSIYDMSWVRQVPGKGLEWVSYI
SSGGGTTYYPDTVKGRFTISRDNSRNTLDLQMNSLRVEDTAVYYCARHSG
YGSSYGVLFAYWGQGTLVTVS
linker
SEQ ID No. 18
SGGGGSGGGGSGGGGS
aCD22 light
SEQ ID No. 25
DIQMTQSPSSLSASVGDRVTITCRASQDISNYLNWLQQKPGKAPKLLIYY
TSILHSGVPSRFSGSGSGTEFTLTISSLQPEDFATYYCQQGNTLPWTFGQ
GTKLEIKRSDPA
HCH2CH3pvaa
SEQ ID No. 26
EPKSPDKTHTCPPCPAPPVAGPSVFLFPPKPKDTLMIARTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGKKDPK
hCD148 TMdomain
SEQ ID No. 27
AVFGCIFGALVIVTVGGFIFW
hCD148 cytosolic domain
SEQ ID No. 28
RKKRKDAKNNEVSFSQIKPKKSKLIRVENFEAYFKKQQADSNCGFAEEYE
DLKLVGISQPKYAAELAENRGKNRYNNVLPYDISRVKLSVQTHSTDDYIN
ANYMPGYHSKKDFIATQGPLPNTLKDFWRMVWEKNVYAIIMLTKCVEQGR
TKCEEYWPSKQAQDYGDITVAMTSEIVLPEWTIRDFTVKNIQTSESHPLR
QFHFTSWPDHGVPDTTDLLINFRYLVRDYMKQSPPESPILVHCSAGVGRT
GTFIAIDRLIYQIENENTVDVYGIVYDLRMHRPLMVQTEDQYVFLNQCVL
DIVRSQKDSKVDLIYQNTTAMTIYENLAPVTTFGKTNGYIA*

A similar construct was also made with the same binding domains and endodomains, but in which the spacer of the CD22 CAR was derived from the murine CD8 stalk (aCD19-huCD8STK-CD28tmZ-2A-aCD22hum-muCD8STK-dCD148).

The sequence of the polypeptide was the same as that for SEQ ID No. 1 except the portion corresponding to HCH2CH3pvaa (SEQ ID No. 26 above) is replaced with muCD8STK (SEQ ID No. 29)

muCD8aSTK
SEQ ID No. 29
TTTKPVLRTPSPVHPTGTSQPQRPEDCRPRGSVKGTGLDFACDIYKDPK

The CAR may comprise a variant of the CAR-encoding part of the sequence shown as SEQ ID No. 1 having at least 80, 85, 90, 95, 98 or 99% sequence identity, provided that the variant sequence is a CAR having the required properties.

Methods of sequence alignment are well known in the art and are accomplished using suitable alignment programs. The % sequence identity refers to the percentage of amino acid or nucleotide residues that are identical in the two sequences when they are optimally aligned. Nucleotide and protein sequence homology or identity may be determined using standard algorithms such as a BLAST program (Basic Local Alignment Search Tool at the National Center for Biotechnology Information) using default parameters, which is publicly available at http://blast.ncbi.nlm.nih.gov. Other algorithms for determining sequence identity or homology include: LALIGN (http://wvvw.ebi.ac.uk/Tools/psa/lalign/ and (http://wvvw.ebi.ac.uk/Tools/psa/lalign/nucleotide.html), AMAS (Analysis of Multiply Aligned Sequences, at http://www.compbio.dundee.ac.uk/Software/Amas/amas.html), FASTA (http://www.ebi.ac.uk/Tools/sss/fasta/), Clustal Omega (http://www.ebi.ac.uk/Tools/msa/clustalo/), SIM (http://web.expasy.org/sim/), and EMBOSS Needle (http://www.ebi.ac.uk/Tools/psa/emboss_needle/nucleotide.html).

### The Car Logical and Gate

In the AND gate of the invention and as described previously in WO2015/075469, one CAR comprises an activating endodomain and one CAR comprises an inhibitory endodomain. The inhibitory CAR constitutively inhibits the first activating CAR but, upon recognition of its cognate antigen, releases its inhibition of the activating CAR. In this manner, a T-cell can be engineered to trigger only if a target cell expresses both cognate antigens. This behaviour is achieved by the activating CAR comprising an activating endodomain containing ITAM domains for example the endodomain of CD3 Zeta, and the inhibitory CAR comprising the endodomain from a phosphatase able to dephosphorylate an ITAM (e.g. CD45 or CD148).

In the system described in WO2015/075469, the spacer domains of both CARs are significantly different in, for example, size. When only the activating CAR is ligated, the inhibitory CAR is in solution on the T-cell surface and can diffuse in and out of the synapse inhibiting the activating CAR. When both CARs are ligated, due to differences in spacer properties, the activating and inhibiting CAR are differentially segregated allowing the activating CAR to trigger T-cell activation unhindered by the inhibiting CAR.

In the system of the present invention, the target epitopes from the activating and inhibitory CARs are positioned a significantly different relative distance from the target cell membrane. For example, the target epitope for the activating CAR may be positioned relatively proximal to the target cell membrane, whereas the target epitope for the inhibitory CAR may be positioned relatively distal to the target cell membrane. Where there is a significant difference in relative position of the target epitopes for the activating and inhibitory CARs, a functional AND gate may be created regardless of the nature of the spacers on the activating and inhibitory CARs. In this new type of AND gate, the spacers of the two (or more) CARs may be are similar or different in terms of size and/or shape and/or charge.

### Signal Peptide

The CARs of the T cell of the present invention may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the SEQ ID No. 2, 3 or 4 or a variant thereof having 5, 4, 3, 2 or 1 amino acid mutations (insertions, substitutions or additions) provided that the signal peptide still functions to cause cell surface expression of the CAR.

| | |
|---|---|
| SEQ ID No. 2: | MGTSLLCWMALCLLGADHADG |

The signal peptide of SEQ ID No. 2 is compact and highly efficient. It is predicted to give about 95% cleavage after the terminal glycine, giving efficient removal by signal peptidase.

| | |
|---|---|
| SEQ ID No. 3: | MSLPVTALLLPLALLLHAARP |

The signal peptide of SEQ ID No. 3 is derived from IgG1.

| | |
|---|---|
| SEQ ID No. 4: | MAVPTQVLGLLLLWLTDARC |

The signal peptide of SEQ ID No. 4 is derived from CD8.

The signal peptide for the first CAR may have a different sequence from the signal peptide of the second CAR (and from the 3^{rd} CAR and 4^{th} CAR etc).

### Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell.

WO2015/052538 describes a CAR which binds BCMA, in which the antigen binding domain comprises APRIL, a ligand for BCMA.

The antigen binding domain may be based on a natural ligand of the antigen.

The antigen binding domain may comprise an affinity peptide from a combinatorial library or a de novo designed affinity protein/peptide.

### Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

In the cell of the present invention, the first and second CARs may comprise the same or different spacer molecules. For example, the spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Examples of amino acid sequences for these spacers are given below:

SEQ ID No. 5 (hinge-CH2CH3 of human IgG1)
AEPKSPDKTHTCPPCPAPPVAGPSVFLFPPKPKDTLMIARTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKKD
SEQ ID No. 6 (human CD8 stalk):
TTTPAPRPPTPAPTIASQPLSLRPEACRPAAGGAVHTRGLDFACDI
SEQ ID No. 7 (human IgG1 hinge):
AEPKSPDKTHTCPPCPKDPK
SEQ ID No. 8 (CD2 ectodomain)
KEITNALETWGALGQDINLDIPSFQMSDDIDDIKWEKTSDKKKIAQFRK
EKETFKEKDTYKLFKNGTLKIKHLKTDDQDIYKVSIYDTKGKNVLEKIF
DLKIQERVSKPKISWTCINTTLTCEVMNGTDPELNLYQDGKHLKLSQRV
ITHKWTTSLSAKFKCTAGNKVSKESSVEPVSCPEKGLD
SEQ ID no. 9 (CD34 ectodomain)
SLDNNGTATPELPTQGTFSNVSTNVSYQETTTPSTLGSTSLHPVSQHGN
EATTNITETTVKFTSTSVITSVYGNTNSSVQSQTSVISTVFTTPANVST
PETTLKPSLSPGNVSDLSTTSTSLATSPTKPYTSSSPILSDIKAEIKCS
GIREVKLTQGICLEQNKTSSCAEFKKDRGEGLARVLCGEEQADADAGAQ
VCSLLLAQSEVRPQCLLLVLANRTEISSKLQLMKKHQSDLKKLGILDFT
EQDVASHQSYSQKT
SEQ ID No. 30 (Human CD28STK)
KIEVMYPPPYLDNEKSNGTIIHVKGKHLCPSPLFPGPSKP
SEQ ID No. 31 (Human TCRz ectodomain):
QSFGLLDPK

Since CARs are typically homodimers (see FIG. 2), cross-pairing may result in a heterodimeric chimeric antigen receptor. This is undesirable for various reasons, for example: (1) the epitope may not be at the same “level” on the target cell so that a cross-paired CAR may only be able to bind to one antigen; (2) the VH and VL from the two different scFv could swap over and either fail to recognize target or worse recognize an unexpected and unpredicted antigen. For the AND gate of the present invention, the spacer of the first CAR may be sufficiently different from the spacer of the second CAR in order to avoid cross-pairing. The amino acid sequence of the first spacer may share less that 50%, 40%, 30% or 20% identity at the amino acid level with the second spacer.

Unlike the AND gate system described in WO2015/075469, in the specific situation where there is a significant difference in relative position of the target epitopes for the activating and inhibitory CARs, a functional AND gate may be created regardless of the nature of the spacers on the activating and inhibitory CARs. In this system it is therefore not important that the spacer of the first CAR has a different length, and/or charge and/or shape and/or configuration and/or glycosylation than the spacer of the second CAR. The difference in relative positions of the target epitopes at the T-cell/target cell synapse alone is enough to ensure that when both first and second CARs bind their target antigen, dys-segregation occurs resulting in spatial separation of the activating and inhibitory CARs to different parts of the membrane.

The present inventors have, in fact, shown that where there is a significant difference in relative position of the target epitopes for the activating and inhibitory CARs, AND gate function may actually be improved by using similarly sized spacers on the activating and inhibitory CARs.

The spacer of the first and second CARs may therefore be of an equivalent size. An “equivalent size” in the context of the present invention means that the two spacers are similar in terms of their length. Two spacers of an equivalent size may cause co-segregation when the pair of CARs target two epitopes which are positioned an equivalent distance from the target cell membrane. For example, where one CAR has an antigen binding domain based on fmc63 which binds CD19; and the other CAR has an antigen binding domain based on P67.7 which binds CD33 a pair of spacers “of equivalent size” would lead to co-segregation of the two CARs.

The spacers of the activating and inhibitory CARs may be similar in size, but sufficiently different to prevent cross-pairing. Pairs of orthologous spacer sequences may be employed, such as murine and human CD8 stalks.

Examples of spacer pairs which are of a relatively similar size are given in the following table:

| Stimulatory CAR spacer | Inhibitory CAR spacer |
|---|---|
| Human-CD8aSTK | Mouse CD8aSTK |
| Human-CD28STK | Mouse CD8aSTK |
| Human-IgG-Hinge | Human-CD3z ectodomain |
| Human-CD8aSTK | Mouse CD28STK |
| Human-CD28STK | Mouse CD28STK |
| Human-IgG-Hinge-CH2CH3 | Human-IgM-Hinge-CH2CH3CD4 |

The spacers in a spacer pair of equivalent size may have a similar number of amino acids. The group of “small” spacers may have, for example between 5 and 100; between 15 and 75; or between 15 and 50 amino acids. Examples of small spacers include human CD8 stalk (46 amino acids), human IgG hinge (20 amino acids), human CD3z ectodomain (9 amino acids), and human CD28 (40 amino acids) and orthologues of these sequences.

The group of “medium sized” spacers may have, for example between 100 and 200 amino acids. Examples of medium sized spacers include CD2 ectodomian (185 amino acids) and orthologues of this sequence.

The group of “large” spacers may have, for example at least 200 amino acids, for example between 100 and 300 amino acids. Examples of large spacers include human IgG hinge CH2CH3 (234 amino acids), human CD34 ectodomain (259 amino acids) and orthologues of these sequences.

In a spacer pair of equivalent size, the two spacers may be from the same size group, i.e. both small, medium sized, or large.

In terms of the number of amino acids in the spacer sequence, one spacer may be within 75%-150% the length of the other spacer.

### Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e. a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good receptor stability.

### Activating Endodomain

The endodomain is the signal-transmission portion of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The cell of the present invention comprises a CAR with an activating endodomain, which may comprise the CD3-Zeta endodomain alone, the CD3-Zeta endodomain with that of either CD28 or OX40 or the CD28 endodomain and OX40 and CD3-Zeta endodomain.

Any endodomain which contains an ITAM motif can act as an activation endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxL/I. Typically, but not always, two of these motifs are separated by between 6 and 8 amino acids in the tail of the molecule (YxxL/Ix(6-8)YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000063372, which relates to synthetic signalling molecules).

The transmembrane and intracellular T-cell signalling domain (endodomain) of a CAR with an activating endodomain may comprise the sequence shown as SEQ ID No. 10, 11 or 12 or a variant thereof having at least 80% sequence identity.

comprising CD28 transmembrane domain and CD3 Z
endodomain
SEQ ID No. 10
FWVLVVVGGVLACYSLLVTVAFIIFWVRRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR
comprising CD28 transmembrane domain and CD28 and
CD3 Zeta endodomains
SEQ ID No. 11
FWVLVVVGGVLACYSLLVTVAFIIFWVRSKRSRLLHSDYMNMTPRRPGPT
RKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR
comprising CD28 transmembrane domain and CD28,
OX40 and CD3 Zeta endodomains.
SEQ ID No. 12
FWVLVVVGGVLACYSLLVTVAFIIFWVRSKRSRLLHSDYMNMTPRRPGPT
RKHYQPYAPPRDFAAYRSRDQRLPPDAHKPPGGGSFRTPIQEEQADAHST
LAKIRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPR

A variant sequence may have at least 80%, 85%, 90%, 95%, 98% or 99% sequence identity to SEQ ID No. 10, 11 or 12, provided that the sequence provides an effective trans-membrane domain and an effective intracellular T cell signaling domain.

### “Ligation-Off” Inhibitory Endodomain

In the AND gate of the present invention, one of the CARs comprises an inhibitory endodomain such that the inhibitory CAR inhibits T-cell activation by the activating CAR in the absence of inhibitory CAR ligation, but does not significantly inhibit T-cell activation by the activating CAR when the inhibitory CAR is ligated. This is termed a “ligation-off” inhibitory endodomain.

When both receptors are ligated, the activating CARs and the inhibitory CARs are isolated in different membrane compartments of the immunological synapse, so that the activating endodomain is released from inhibition by the inhibitory endodomain.

The inhibitory endodomains for use in a ligation-off inhibitory CAR may therefore comprise any sequence which inhibits T-cell signaling by the activating CAR when it is in the same membrane compartment (i.e. in the absence of the antigen for the inhibitory CAR) but which does not significantly inhibit T cell signaling when it is isolated in a separate part of the membrane from the inhibitory CAR.

The ligation-off inhibitory endodomain may be or comprise a tyrosine phosphatase, such as a receptor-like tyrosine phosphatase. An inhibitory endodomain may be or comprise any tyrosine phosphatase that is capable of inhibiting the TCR signalling when only the stimulatory receptor is ligated. An inhibitory endodomain may be or comprise any tyrosine phosphatase with a sufficiently fast catalytic rate for phosphorylated ITAMs that is capable of inhibiting the TCR signalling when only the stimulatory receptor is ligated.

For example, the inhibitory endodomain of an AND gate may comprise the endodomain of CD148 or CD45. CD148 and CD45 have been shown to act naturally on the phosphorylated tyrosines up-stream of TCR signalling.

CD148 is a receptor-like protein tyrosine phosphatase which negatively regulates TCR signaling by interfering with the phosphorylation and function of PLCγ1 and LAT.

CD45 present on all hematopoetic cells, is a protein tyrosine phosphatase which is capable of regulating signal transduction and functional responses, again by phosphorylating PLC γ1.

An inhibitory endodomain may comprise all of part of a receptor-like tyrosine phosphatase. The phospatase may interfere with the phosphorylation and/or function of elements involved in T-cell signalling, such as PLCγ1 and/or LAT.

The transmembrane and endodomain of CD45 and CD148 is shown as SEQ ID No. 13 and No. 14 respectively.

CD45 trans-membrane and endodomain sequence
SEQ ID 13
ALIAFLAFLIIVTSIALLVVLYKIYDLHKKRSCNLDEQQELVERDDEKQ
LMNVEPIHADILLETYKRKIADEGRLFLAEFQSIPRVFSKFPIKEARKP
FNQNKNRYVDILPYDYNRVELSEINGDAGSNYINASYIDGFKEPRKYIA
AQGPRDETVDDFWRMIWEQKATVIVMVTRCEEGNRNKCAEYWPSMEEGT
RAFGDVVVKINQHKRCPDYIIQKLNIVNKKEKATGREVTHIQFTSWPDH
GVPEDPHLLLKLRRRVNAFSNFFSGPIVVHCSAGVGRTGTYIGIDAMLE
GLEAENKVDVYGYVVKLRRQRCLMVQVEAQYILIHQALVEYNQFGETEV
NLSELHPYLHNMKKRDPPSEPSPLEAEFQRLPSYRSWRTQHIGNQEENK
SKNRNSNVIPYDYNRVPLKHELEMSKESEHDSDESSDDDSDSEEPSKYI
NASFIMSYWKPEVMIAAQGPLKETIGDFWQMIFQRKVKVIVMLTELKHG
DQEICAQYWGEGKQTYGDIEVDLKDTDKSSTYTLRVFELRHSKRKDSRT
VYQYQYTNWSVEQLPAEPKELISMIQVVKQKLPQKNSSEGNKHHKSTPL
LIHCRDGSQQTGIFCALLNLLESAETEEVVDIFQVVKALRKARPGMVST
FEQYQFLYDVIASTYPAQNGQVKKNNHQEDKIEFDNEVDKVKQDANCVN
PLGAPEKLPEAKEQAEGSEPTSGTEGPEHSVNGPASPALNQGS
CD148 trans-membrane and endodomain sequence
SEQ ID 14
AVFGCIFGALVIVTVGGFIFWRKKRKDAKNNEVSFSQIKPKKSKLIRVE
NFEAYFKKQQADSNCGFAEEYEDLKLVGISQPKYAAELAENRGKNRYNN
VLPYDISRVKLSVQTHSTDDYINANYMPGYHSKKDFIATQGPLPNTLKD
FWRMVWEKNVYAIIMLTKCVEQGRTKCEEYWPSKQAQDYGDITVAMTSE
IVLPEWTIRDFTVKNIQTSESHPLRQFHFTSWPDHGVPDTTDLLINFRY
LVRDYMKQSPPESPILVHCSAGVGRTGTFIAIDRLIYQIENENTVDVYG
IVYDLRMHRPLMVQTEDQYVFLNQCVLDIVRSQKDSKVDLIYQNTTAMT
IYENLAPVTTFGKTNGYIA

An inhibitory CAR may comprise all or part of SEQ ID No 13 or 14 (for example, it may comprise the phosphatase function of the endodomain). It may comprise a variant of the sequence or part thereof having at least 80% sequence identity, as long as the variant retains the capacity to basally inhibit T cell signalling by the activating CAR.

Other spacers and endodomains may be tested for example using the model system exemplified herein. Target cell populations can be created by transducing a suitable cell line such as a SupT1 cell line either singly or doubly to establish cells negative for both antigens (the wild-type), positive for either and positive for both (e.g. CD19−CD33−, CD19+CD33−, CD19−CD33+ and CD19+CD33+). T cells such as the mouse T cell line BW5147 which releases IL-2 upon activation may be transduced with pairs of CARs and their ability to function in a logic gate measured through measurement of IL-2 release (for example by ELISA).

### Differentially Positioned Target Epitopes

In the cell of the present invention, the first (activatory) CAR binds to a first antigen and the second (inhibitory) CAR binds to a second antigen; both the first and second antigens being expressed on the surface of a target cell.

The activatory CAR binds to an epitope on the first antigen which is relatively proximal to the membrane of the target cell, the inhibitory CAR binds to an epitope on the second antigen which is relatively distal to the membrane of the target cell. Thus the epitope on the first antigen is relatively closer to the target cell membrane, for example tumour cell membrane, than the epitope on the second antigen.

The relative distances at a T-cell:target cell synapse are illustrated schematically in FIG. 5, in which:

X is the distance from the target cell membrane to the epitope on the first antigen;
X1 is the length of the first CAR;
Y is the distance from the target cell membrane to the epitope on the second antigen;
Y1 is the length of the second CAR.

In the AND gate system of the invention, Y>>X.

X1 and Y1 may be approximately equal.

When the first CAR and the second CAR bind their respective target antigens,

(X+X1)«(Y+Y1)

such that the first CAR and second CAR become spatially separated on the immune cell membrane due to kinetic segregation.

The epitope of the first antigen is relatively proximal to the target cell membrane, whereas the epitope of the second antigen is relatively distal to the target cell membrane. The distance from the target cell membrane to the epitope on the second antigen (Y) may be at least 1.5 times the distance from the target cell membrane to the epitope on the first antigen. Y may be between 1.5 and 3 times X. Y may be approximately 2 times X.

X+X1 may be approximately 15 nm, for example, between 12-18 nm.

Y+Y1 may be greater than 20 nm, for example between 20 and 30 nm.

X may be less than or approximately equal to 8 nm, for example between 5 and 8 nm. Y may be greater than or approximately equal to 15 nm, for example between 15 and 25 nm.

X may be approximately equivalent to the length of two Ig domains, whereas Y may be approximately equivalent to the length of four, five or more Ig domains.

The terms “relatively distal” and “relatively proximal” are used to indicate that there is a significant difference in the relative distance of the two epitopes from the target cell membrane, significant enough to cause segregation of the two CARs at the T-cell:target cell synapse when both CARs bind their target antigen, when the two CARs have spacers of an equivalent size (such as orthologous spacers).

The present invention is readily applied to AND gate approaches targeting a pair of antigens which are of different sizes, i.e. which extend by different distances from the target cell membrane. This enables differentially spaced epitopes to be selected which are near the “end” of both molecules, and thus accessible to CAR binding, rather than having to select one epitope which is buried within the antigen molecule.

The AND gate of the present invention functions best if the distal epitope is targeted by the inhibitory CAR. Examples of antigens having long extracellular portions which are suitable for targeting with an inhibitory CAR in the AND gate of the invention include MUC16, CEACAMS, MUC1, CD22, NCAM1 and Chondroitin sulfate proteoglycan 4.

The following targets are “mid” range in size: PSMA, EpCAM, EGFR, IGF1R, EBB2, Integrin alpha-V/beta-3. These may also be suitable for use with an AND gate of the invention as the “distal” epitope.

For the “proximal” epitope, one option is to select an antigen with a relatively short extracellular domain, such as: CD20, GD2/3, CD80, CD23, CD52, CD319, FOLR1, CD33, CD75, CD38, CD19, CD2, PSCA, IL6R, IL15R, IL2R, FCGR1 or HLA-DR.

An alternative option, is to target a membrane proximal epitope on an antigen of any size i.e. including antigens with mid-range and long extracellular portions.

### Co-Expression Site

The second aspect of the invention relates to a nucleic acid which encodes the first and second CARs.

The nucleic acid may produce a polypeptide which comprises the two CAR molecules joined by a cleavage site. The cleavage site may be self-cleaving, such that when the polypeptide is produced, it is immediately cleaved into the first and second CARs without the need for any external cleavage activity.

Various self-cleaving sites are known, including the Foot-and-Mouth disease virus (FMDV) 2a self-cleaving peptide, which has the sequence shown as SEQ ID No. 15:

SEQ ID No. 15
RAEGRGSLLTCGDVEENPGP.

The co-expressing sequence may be an internal ribosome entry sequence (IRES).

The co-expressing sequence may be an internal promoter.

### Cell

The first aspect of the invention relates to a cell which co-expresses a first CAR and a second CAR at the cell surface.

The cell may be any eukaryotic cell capable of expressing a CAR at the cell surface, such as an immunological cell.

In particular the cell may be an immune effector cell such as a T cell or a natural killer (NK) cell

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The T cell of the invention may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

CAR-expressing cells, such as CAR-expressing T or NK cells, may either be created ex vivo either from a patient's own peripheral blood (1^{st} party), or in the setting of a haematopoietic stem cell transplant from donor peripheral blood (2^{nd} party), or peripheral blood from an unconnected donor (3^{rd} party).

The present invention also provide a cell composition comprising CAR expressing T cells and/or CAR expressing NK cells according to the present invention. The cell composition may be made by tranducing or transfecting a blood-sample ex vivo with a nucleic acid according to the present invention.

Alternatively, CAR-expressing cells may be derived from ex vivo differentiation of inducible progenitor cells or embryonic progenitor cells to the relevant cell type, such as T cells. Alternatively, an immortalized cell line such as a T-cell line which retains its lytic function and could act as a therapeutic may be used.

In all these embodiments, CAR cells are generated by introducing DNA or RNA coding for the CARs by one of many means including transduction with a viral vector, transfection with DNA or RNA.

A CAR T cell of the invention may be an ex vivo T cell from a subject. The T cell may be from a peripheral blood mononuclear cell (PBMC) sample. T cells may be activated and/or expanded prior to being transduced with CAR-encoding nucleic acid, for example by treatment with an anti-CD3 monoclonal antibody.

A CAR T cell of the invention may be made by:
(i) isolation of a T cell-containing sample from a subject or other sources listed above; and
(ii) transduction or transfection of the T cells with one or more nucleic acid sequence(s) encoding the first and second CAR.

The T cells may then by purified, for example, selected on the basis of co-expression of the first and second CAR.

### Nucleic Acid Sequences

The second aspect of the invention relates to one or more nucleic acid sequence(s) which codes for a first CAR and a second CAR as defined in the first aspect of the invention.

The nucleic acid sequence may be DNA or RNA.

### Vector

The present invention also provides a vector, or kit of vectors which comprises one or more CAR-encoding nucleic acid sequence(s). Such a vector may be used to introduce the nucleic acid sequence(s) into a host cell so that it expresses the first and second CARs.

The vector may, for example, be a plasmid or a viral vector, such as a retroviral vector or a lentiviral vector, or a transposon based vector or synthetic mRNA.

The vector may be capable of transfecting or transducing a T cell.

### Pharmaceutical Composition

The present invention also relates to a pharmaceutical composition containing a plurality of CAR-expressing cells, such as T cells or NK cells according to the first aspect of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The cells of the present invention may be capable of killing target cells, such as cancer cells. The target cell may be recognisable by a defined pattern of antigen expression, for example the expression of antigen A AND antigen B; or the expression of antigen A AND antigen B AND antigen C.

The cells of the present invention may be used for the treatment of an infection, such as a viral infection.

The cells of the invention may also be used for the control of pathogenic immune responses, for example in autoimmune diseases, allergies and graft-vs-host rejection.

The cells of the invention may be used for the treatment of a cancerous disease, such as bladder cancer, breast cancer, colon cancer, endometrial cancer, kidney cancer (renal cell), leukemia, lung cancer, melanoma, non-Hodgkin lymphoma, pancreatic cancer, prostate cancer and thyroid cancer.

It is particularly suited for treatment of solid tumours where the availability of good selective single targets is limited.

The cells of the invention may be used to treat: cancers of the oral cavity and pharynx which includes cancer of the tongue, mouth and pharynx; cancers of the digestive system which includes oesophageal, gastric and colorectal cancers; cancers of the liver and biliary tree which includes hepatocellular carcinomas and cholangiocarcinomas; cancers of the respiratory system which includes bronchogenic cancers and cancers of the larynx; cancers of bone and joints which includes osteosarcoma; cancers of the skin which includes melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers including gliomas, glioblastoma multiforme and medullobastomas; cancers of the endocrine system including thyroid cancer, adrenal carcinoma and cancers associated with multiple endocrine neoplasm syndromes; lymphomas including Hodgkin's lymphoma and non-Hodgkin lymphoma; Multiple Myeloma and plasmacytomas; leukaemias both acute and chronic, myeloid or lymphoid; and cancers of other and unspecified sites including neuroblastoma.

Treatment with the cells of the invention may help prevent the escape or release of tumour cells which often occurs with standard approaches.

The invention will now be further described by way of Examples, which are meant to serve to assist one of ordinary skill in the art in carrying out the invention and are not intended in any way to limit the scope of the invention.

### EXAMPLES

### Example 1—Investigating AND Gate Function with an Extended Target Antigen

As described in WO2015/075469, an AND gate combines a simple activating receptor with a receptor which basally inhibits activity, but whose inhibition is turned off once the receptor is ligated. In the examples described in WO2015/075469, this was achieved for an CD19/CD33 target antigen pair (and various other antigen combinations) by combining a standard 1^{st} generation CAR with a short/non-bulky CD8 stalk spacer and a CD3 Zeta endodomain with a second receptor with a bulky Fc spacer whose endodomain contained either CD148 or CD45 endodomains. When both receptors are ligated, the difference in spacer dimensions results in isolation of the different receptors in different membrane compartments, releasing the CD3 Zeta receptor from inhibition by the CD148 or CD45 endodomains. In this way, activation only occurs once both receptors are activated.

In order to investigate the effect of distance of the epitopes from the target cell membrane on AND gate function, a set of target cells were created expressing a truncation series of CD22 (FIG. 3A). CD22 has a very long extracellular portion containing seven immunoglobulin domains. The binding site for RFB-4 is in the fifth Ig domain from the membrane. A series of deletion mutants were created lacking Ig domains 1 and 2 (CD22-2Ig); Ig domains, 1, 2 and 3 (CD22-3Ig); or Ig domains 1, 2, 3 and 4 (CD22-4Ig), bringing the RFB-4 binding epitope incrementally closer to the target cell membrane.

T cells were created expressing 1) and anti-CD19 activating CAR with a CD8 stalk spacer and a CD3 Zeta endodomain, and 2) an anti-CD22 inhibitory CAR with an RFB-4-based antigen-binding domain, an Fc spacer and a CD148 endodomain.

AND gate function was tested and it was shown that, for all truncated CD22 variants, the T-cell was only activated in the presence of both antigens (FIG. 3B). So all truncated CD22 variants produced a functional AND gate.

Importantly, however, it was found that AND gate function was improved by increasing the distance of the RFB-4 binding epitope from the target cell membrane (FIG. 4B), so that although the shortest variant (CD22-4Ig) produced a functional AND gate, the target antigen in which the RFB-4 binding epitope is positioned further from the target cell membrane (CD22 “original”) gave the best AND gate function.

### Example 2—AND Gate Function with Differentially Positioned CAR Epitopes

AND gate function was then investigated for a target antigen pair where there is a significant difference in relative spacing of the epitopes from the target cell membrane, but this time using spacers of a similar size on the activating and inhibitory CAR.

T cells were created expressing 1) and anti-CD19 activating CAR with a human CD8 stalk spacer and a CD3 Zeta endodomain, and 2) an anti-CD22 inhibitory CAR with an RFB-4-based antigen-binding domain, a murine CD8 stalk spacer and a CD148 endodomain (FIG. 4A). The murine and human CD8 stalk spacers are orthologous and are of a similar length.

The function of T cells expressing this AND gate CAR pair was compared with that of T cells expressing the AND gate pair described in Example 1, in which the activating CAR comprises a CD8 stalk spacer, and the inhibitor CAR comprises an Fc spacer (FIG. 4A).

As shown in FIG. 4B, it was found that both CAR pairs functioned as an AND gate. However, surprisingly, for a target antigen pair where there is a significant difference in spacing of the epitopes from the target cell membrane, AND gate function was improved using a pair of CARs with similar sized spacers, compared to a CAR pair where the activating CAR comprises a short/non-bulky spacer and the inhibitory CAR comprises a long/bulky spacer.

This is particularly surprising in view of the data shown in Example 6 of WO2015/075469 which shows that for an antigen pair where the epitopes are positioned a similar distance from the target cell membrane (CD19 and CD33) using a pair of CARs with orthologous murine/human CD8 stalk spacers abolishes AND gate function.

In situations where it is possible to select a pair of epitopes which are positioned at significantly different distances from the target cell membrane, for example, where the two (or more) antigens targeted in the AND gate are of a significantly different size, kinetic segregation may be induced to occur based on this different distance. In this situation it is no longer necessary to use spacers of different length/bulkiness on the activating and inhibitory CARs. Indeed, AND gate function can be improved in this situation by using similar spacers on the activating and inhibitory CARs.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which co-expresses a first chimeric antigen receptor (CAR) and second CAR at the cell surface, each CAR comprising:
(i) an antigen-binding domain;
(ii) a spacer
(iii) a trans-membrane domain; and
(iv) an endodomain
wherein the first CAR is an activating CAR comprising an activating endodomain and the second CAR is an inhibitory CAR comprising a ligation-off inhibitory endodomain;
wherein the first CAR binds to a first antigen and the second CAR binds to a second antigen, wherein both the first and second antigens are expressed on the surface of a target cell;
wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
wherein the spacer of the first CAR is an equivalent size to the spacer of the second CAR.

2. A cell according to claim 1, wherein, when the first CAR and the second CAR bind their respective target antigens,
(X+X1)«(Y+Y1)
where:
X is the distance from the target cell membrane to the epitope on the first antigen;
X1 is the length of the first CAR;
Y is the distance from the target cell membrane to the epitope on the second antigen;
Y1 is the length of the second CAR;
such that the first CAR and second CAR become spatially separated on the immune cell membrane.

3. A cell according to claim 2, wherein X is less than 8 nm and Y is greater than 15 nm.

4. A cell according to claim 1, wherein the spacer of the first CAR and the spacer of the second CAR are orthologous.

5. A cell according to claim 1, wherein the inhibitory endodomain comprises all or part of the endodomain from CD148 or CD45.

6. A cell according to claim 1 which is a T-cell or a natural killer (NK) cell.

7. A nucleic acid sequence encoding a first chimeric antigen receptor (CAR) and a second CAR, each CAR comprising:
(i) an antigen-binding domain;
(ii) a spacer
(iii) a trans-membrane domain; and
(iv) an endodomain
wherein the first CAR is an activating CAR comprising an activating endodomain and the second CAR is an inhibitory CAR comprising a ligation-off inhibitory endodomain;
wherein the first CAR binds to a first antigen and the second CAR binds to a second antigen, wherein both the first and second antigens are expressed on the surface of a target cell;
wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
wherein the spacer of the first CAR is an equivalent size to the spacer of the second CAR.

8. A nucleic acid sequence according to claim 7, which has the following structure:
AgB1-spacer1-TM1-endo1-coexpr-AbB2-spacer2-TM2-endo2
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the endodomain of the first CAR;
coexpr is a nucleic acid sequence enabling co-expression of both CARs
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the endodomain of the second CAR;
which nucleic acid sequence, when expressed in a cell, encodes a polypeptide which is cleaved at the cleavage site such that the first and second CARs are co-expressed at the cell surface.

9. A nucleic acid sequence according to claim 8, wherein coexpr encodes a sequence comprising a self-cleaving peptide.

10. A nucleic acid sequence according to claim 8 or 9, wherein alternative codons are used in regions of sequence encoding the same or similar amino acid sequences, in order to avoid homologous recombination.

11. A kit of nucleic acid sequences encoding a first chimeric antigen receptor (CAR) and second CAR, each CAR comprising:
(i) an antigen-binding domain;
(ii) a spacer
(iii) a trans-membrane domain; and
(iv) an endodomain
wherein the first CAR is an activating CAR comprising an activating endodomain and the second CAR is an inhibitory CAR comprising a ligation-off inhibitory endodomain;
wherein the first CAR binds to a first antigen and the second CAR binds to a second antigen, wherein both the first and second antigens are expressed on the surface of a target cell;
wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
wherein the spacer of the first CAR is an equivalent size to the spacer of the second CAR,
wherein the kit comprises
(i) a first nucleic acid sequence encoding the first chimeric antigen receptor (CAR), which nucleic acid sequence has the following structure:
AgB1-spacer1-TM1-endo1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the endodomain of the first CAR; and
(ii) a second nucleic acid sequence encoding the second chimeric antigen receptor (CAR), which nucleic acid sequence has the following structure:
AgB2-spacer2-TM2-endo2
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the endodomain of the second CAR.

12. A kit of vectors for producing a first chimeric antigen receptor (CAR) and second CAR, each CAR comprising:
(i) an antigen-binding domain;
(ii) a spacer
(iii) a trans-membrane domain; and
(iv) an endodomain
wherein the first CAR is an activating CAR comprising an activating endodomain and the second CAR is an inhibitory CAR comprising a ligation-off inhibitory endodomain;
wherein the first CAR binds to a first antigen and the second CAR binds to a second antigen, wherein both the first and second antigens are expressed on the surface of a target cell;
wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
wherein the spacer of the first CAR is an equivalent size to the spacer of the second CAR
wherein the kit comprises:
a) a first vector which comprises a first nucleic acid sequence encoding the first CAR; and
b) a second vector which comprises a second nucleic acid sequence encoding the second CAR.

13. (canceled)

14. A vector comprising a nucleic acid sequence according to claim 7.

15. A retroviral vector or a lentiviral vector or a transposon according to claim 14.

16. A method for making a cell according claim 1, which comprises the step of introducing into a cell:
(i) a nucleic acid sequence encoding a first chimeric antigen receptor (CAR) and a second CAR, each CAR comprising:
(a) an antigen-binding domain;
(b) a spacer
(c) a trans-membrane domain; and
(d) an endodomain
wherein the first CAR is an activating CAR comprising an activating endodomain and the second CAR is an inhibitory CAR comprising a ligation-off inhibitory endodomain;
wherein the first CAR binds to a first antigen and the second CAR binds to a second antigen, wherein both the first and second antigens are expressed on the surface of a target cell;
wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
wherein the spacer of the first CAR is an equivalent size to the spacer of the second CAR;
(ii) a first nucleotide sequence encoding the first CAR of (i) and a second nucleotide sequence encoding the second CAR of (i);
(iii) a first vector comprising the first nucleotide sequence of (i) and a second vector comprising the second nucleotide sequence of (i); or
(iv) a vector encoding a nucleic acid sequence encoding a first CAR and a second CAR, each CAR comprising:
(a) an antigen-binding domain;
(c) a spacer
(c) a trans-membrane domain; and
(d) an endodomain
wherein the first CAR is an activating CAR comprising an activating endodomain and the second CAR is an inhibitory CAR comprising a ligation-off inhibitory endodomain:
wherein the first CAR binds to a first antigen and the second CAR binds to a second antigen, wherein both the first and second antigens are expressed on the surface of a target cell:
wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
wherein the spacer of the first CAR is an equivalent size to the spacer of the second CAR.

17. A method according to claim 16, wherein the cell is from a sample isolated from a subject.

18. A pharmaceutical composition comprising a plurality of cells according to claim 1.

19. A method for treating and/or preventing a disease, which comprises the step of administering a pharmaceutical composition according to claim 18 to a subject.

20. A method according to claim 19, which comprises the following steps:
(i) isolation of a cell-containing sample from a subject;
(ii) transduction or transfection of the cells with:
(1) a nucleic acid sequence encoding a first chimeric antigen receptor (CAR) and a second CAR, each CAR comprising:
(a) an antigen-binding domain;
(b) a spacer
(c) a trans-membrane domain; and
(d) an endodomain
wherein the first CAR is an activating CAR comprising an activating endodomain and the second CAR is an inhibitory CAR comprising a ligation-off inhibitory endodomain;
wherein the first CAR binds to a first antigen and the second CAR binds to a second antigen, wherein both the first and second antigens are expressed on the surface of a target cell;
wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
wherein the spacer of the first CAR is an equivalent size to the spacer of the second CAR;
(2) a first nucleotide sequence encoding the first CAR of (1) and a second nucleotide sequence encoding the second CAR of (1);
(3) a first vector comprising the first nucleotide sequence of (1) and a second vector comprising the second nucleotide sequence of (1); or
(4) a vector encoding a nucleic acid sequence encoding a first CAR and a second CAR, each CAR comprising:
(a) an antigen-binding domain;
(c) a spacer
(c) a trans-membrane domain; and
(d) an endodomain
wherein the first CAR is an activating CAR comprising an activating endodomain and the second CAR is an inhibitory CAR comprising a ligation-off inhibitory endodomain;
wherein the first CAR binds to a first antigen and the second CAR binds to a second antigen, wherein both the first and second antigens are expressed on the surface of a target cell;
wherein the first CAR binds to an epitope on the first antigen which is relatively proximal to the membrane of the target cell, the second CAR binds to an epitope on the second antigen which is relatively distal to the membrane of the target cell, in comparison with the epitope of the first antigen; and
wherein the spacer of the first CAR is an equivalent size to the spacer of the second CAR; and
(iii) administering the cells from (ii) to a the subject.

21. A method according to claim 19, wherein the disease is a cancer.

22-23. (canceled)


SEQUENCE LISTING (32 sequences)

SEQ ID NO: 1 - protein, 1344 aa, Artificial Sequence
  CAR AND gate which recognizes CD19 AND CD22 using a CD148 phosphatase
MSLPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMEFGLSWLFLVAILKGVQCEVQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVSSGGGGSGGGGSGGGGSDIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKAVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 2 - protein, 21 aa, Artificial Sequence
  signal peptide
MGTSLLCWMALCLLGADHADG

SEQ ID NO: 3 - protein, 21 aa, Artificial Sequence
  signal peptide derived from IgG1
MSLPVTALLLPLALLLHAARP

SEQ ID NO: 4 - protein, 20 aa, Artificial Sequence
  signal peptide derived from CD8
MAVPTQVLGLLLLWLTDARC

SEQ ID NO: 5 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 6 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 7 - protein, 20 aa, Artificial Sequence
  spacer sequence, human IgG1 hinge
AEPKSPDKTHTCPPCPKDPK

SEQ ID NO: 8 - protein, 185 aa, Artificial Sequence
  spacer sequence, CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 9 - protein, 259 aa, Artificial Sequence
  spacer sequence, CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 10 - protein, 140 aa, Artificial Sequence
  comprising CD28 transmembrane domain and CD3 Z endodomain
FWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 180 aa, Artificial Sequence
  comprising CD28 transmembrane domain and CD28 and CD3 Zeta endodomains
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 216 aa, Artificial Sequence
  comprising CD28 transmembrane domain and CD28, OX40 and CD3 Zeta endodomains
FWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 729 aa, Artificial Sequence
  CD45 trans-membrane and endodomain sequence
ALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 14 - protein, 362 aa, Artificial Sequence
  CD148 trans-membrane and endodomain sequence
AVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 15 - protein, 20 aa, Artificial Sequence
  2A peptide
RAEGRGSLLTCGDVEENPGP

SEQ ID NO: 16 - protein, 18 aa, Artificial Sequence
  signal peptide aCD19 CAR
MSLPVTALLLPLALLLHA

SEQ ID NO: 17 - protein, 116 aa, Artificial Sequence
  aCD19 heavy
ARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGG

SEQ ID NO: 18 - protein, 16 aa, Artificial Sequence
  linker
SGGGGSGGGGSGGGGS

SEQ ID NO: 19 - protein, 121 aa, Artificial Sequence
  aCD19 light
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSD

SEQ ID NO: 20 - protein, 47 aa, Artificial Sequence
  CD8STK
PTTTPAPRPPTPAPTIASQPLSLRPEACRPAAGGAVHTRGLDFACDI

SEQ ID NO: 21 - protein, 27 aa, Artificial Sequence
  CD28tm
FWVLVVVGGVLACYSLLVTVAFIIFWV

SEQ ID NO: 22 - protein, 113 aa, Artificial Sequence
  TCRz
RRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 20 aa, Artificial Sequence
  signal peptide aCD22 CAR
MEFGLSWLFLVAILKGVQCE

SEQ ID NO: 24 - protein, 121 aa, Artificial Sequence
  aCD22-heavy
VQLVESGGGLVQPGGSLRLSCAASGFAFSIYDMSWVRQVPGKGLEWVSYISSGGGTTYYPDTVKGRFTISRDNSRNTLDLQMNSLRVEDTAVYYCARHSGYGSSYGVLFAYWGQGTLVTVS

SEQ ID NO: 25 - protein, 112 aa, Artificial Sequence
  aCD22 light
DIQMTQSPSSLSASVGDRVTITCRASQDISNYLNWLQQKPGKAPKLLIYYTSILHSGVPSRFSGSGSGTEFTLTISSLQPEDFATYYCQQGNTLPWTFGQGTKLEIKRSDPA

SEQ ID NO: 26 - protein, 235 aa, Artificial Sequence
  HCH2CH3pvaa
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 27 - protein, 21 aa, Artificial Sequence
  hCD148 TM domain
AVFGCIFGALVIVTVGGFIFW

SEQ ID NO: 28 - protein, 341 aa, Artificial Sequence
  hCD148 cytosolic domain
RKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 29 - protein, 49 aa, Artificial Sequence
  muCD8aSTK
TTTKPVLRTPSPVHPTGTSQPQRPEDCRPRGSVKGTGLDFACDIYKDPK

SEQ ID NO: 30 - protein, 40 aa, Artificial Sequence
  spacer sequence, human CD28STK
KIEVMYPPPYLDNEKSNGTIIHVKGKHLCPSPLFPGPSKP

SEQ ID NO: 31 - protein, 9 aa, Artificial Sequence
  spacer sequence, human TCRz ectodomain
QSFGLLDPK

SEQ ID NO: 32 - protein, 4 aa, Artificial Sequence
  ITAM motif
  misc_feature (2)..(3): Xaa can be any naturally occurring amino acid
  MISC_FEATURE (4)..(4): Xaa may be Leu or Ile
YXXX